(19) United States
(12) Patent Application Publication
(10) Pub. No.: US 2011/0033586 A1
(43) Pub. Date: Feb. 10, 2011

(54) Method For Preparing A Flavonoid Slurry

(75) Inventors: Vincent E. A. Rinaldi, Bethel, CT (US); Craig Hutson, Mundelein, IL (US); Kevin Evans, Cary, IL (US)

Correspondence Address: BANNER & WITCOFF, LTD.;and ATTORNEYS FOR CLIENT NO. 006943, 10 SOUTH WACKER DR., SUITE 3000, CHICAGO, IL, 60606, US

(73) Assignee: Stokely-Van Camp, Inc., Chicago, IL (US)

(21) Appl. No.: 12/853,398

(22) Filed: Aug. 10, 2010

Related U.S. Application Data
(60) Provisional application No. 61/232,490, filed on Aug. 10, 2009.

(51) Int. Cl. A23L 3/3481; B01F 3/12; A23P 1/00; G01N 33/02; A23L 1/27

(52) U.S. Cl. 426/231; 366/348; 426/238; 426/321; 426/250; 426/546

(57) ABSTRACT

A flavonoid slurry and a method for preparing a flavonoid slurry comprising the steps of combining water and flavonoid; mixing the water and flavonoid to obtain a flavonoid-water mixture; and homogenizing the mixture to obtain a flavonoid-water slurry.

### CROSS-REFERENCE TO RELATED APPLICATIONS

This application claims priority to U.S. Provisional Patent Application No. 61/232,490 filed on Aug. 10, 2009, the disclosure of which is expressly incorporated herein by reference.

### FIELD OF THE INVENTION

The present invention relates generally to preparation of a quercetin slurry. More particularly, the present invention relates to a method for preparing a quercetin slurry via homogenization.

### BACKGROUND

Flavonols, in general, are used as nutritional supplements to provide, for example, antioxidants. Quercetin, in particular, is considered a powerful antioxidant. Quercetin is a natural, plant-derived flavonoid. In particular, quercetin is the aglycone form of a number of other flavonoid glycosides, such as rutin and quercitrin, found in citrus fruit, cranberries, blueberries, buckwheat, onions, and other vegetables, fruits, and green plants. The chemical structure of quercetin is illustrated below:

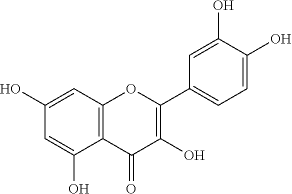

### 2-(3,4-dihydroxyphenyl)-3,5,7-trihydroxy-4H-chromen-4-one

It is desired to use quercetin in food products such as beverages. However, quercetin, which is typically obtained as a powder, is insoluble in water and therefore difficult to disperse and mix into products. When added to liquid media, quercetin usually agglomerizes and settles to the bottom of the beverage, thereby resulting in a product that is not visually appealing to the consumer.

Therefore, a need exists in the food and beverage industry to provide the consumer with a food product containing quercetin wherein the quercetin is easily dispersed and suspended in the product.

### BRIEF SUMMARY

The present invention relates to quercetin slurries. In particular, the present invention relates to a method for preparing a quercetin slurry by homogenizing a quercetin-water mixture. A quercetin slurry can also be prepared by other suitable methods that impart a high level of energy or shear that results in particle size reduction, such as ultrasonic processing.

It was discovered that a quercetin slurry prepared by homogenization provides a stable dispersion of quercetin particles in water. Such slurries will be useful in many applications, in particular, for use in foods and beverages.

### DETAILED DESCRIPTION OF THE INVENTION

The present invention relates to quercetin slurries. In one aspect, the present invention relates to a method for preparing a quercetin slurry by homogenizing a quercetin-water mixture.

In another aspect, the present invention relates to a method for preparing a quercetin slurry by mixing a quercetin-water mixture under high shear and/or ultrasonic processing.

The quercetin may be obtained from any suitable source. The quercetin is typically obtained in dry particle or powdered crystalline form. Typical powdered quercetin has particle sizes ranging between 200 and 800 microns. However, one of ordinary skill in the art would appreciate that various particle sizes may also be used.

In accordance with one aspect of the present invention, quercetin is combined with water and mixed until the quercetin is fully dispersed in the water. The water is preferably distilled water or purified water such as water treated by reverse osmosis. The water is maintained at any suitable temperature, typically around room temperature.

The water and quercetin are mixed for a time sufficient to disperse the quercetin in the water, for example for at least 5 minutes.

After mixing, the water-quercetin mixture is homogenized to obtain a slurry. The mixture may be homogenized in any suitable manner to obtain the desired product. Suitable homogenizers may function by forcing the mixture through very narrow channels or orifices under pressure. The orifices have a diameter to provide the desired particle size reduction. The pressure during homogenization is typically between 3000 psi and 20,000 psi, for example between 5000 psi and 7000 psi. One or two passes through the homogenizer may be used. If two passes are used, the pressure for each pass may be the same or different. Any suitable homogenizer may be used such as those sold by APV or Tetra.

During homogenization, the particle size of the quercetin is reduced. The microparticularized quercetin in the resulting slurry has an average particle size less than 10 microns, in particular less than 3 microns or less than 1 micron. For example, at least 95% of the particles have a particle size less than 20 microns and 80% of the particles have a particle size less than 3 microns. In another aspect, at least 95% of the particles have a particle size less than 10 microns and at least 80% of the particles have a particle size less than 1 micron. The reduction of particle size (or microparticulation) of the quercetin during homogenization provides a stable slurry. Due to density differences between the quercetin particles and water, eventually the quercetin particles will settle.

The slurry may also be prepared by mixing the quercetin and water at pressures below 3000 psi, however more than two passes may be required and/or the particle size may not be as small as desired.

The slurry allows easy dispersion of the quercetin particles in products such as beverages, chews, or bars.

Any suitable amounts of quercetin and water are contemplated, but generally the slurry will contain about 2.5 wt % to about 10 wt % quercetin based on total weight of the slurry. For example, the slurry may contain about 2.5 to about 7 wt %, 2.5 to about 6 wt %, 2.5 wt % to about 5 wt % quercetin, or about 2.5 wt % to about 3 wt % quercetin.

The slurry may also contain organic or inorganic food grade acidulants. These food grade acidulants may include citric acid, ascorbic acid, phosphoric acid, lactic acid, malic acid, fumaric acid, and tartaric acid. One of ordinary skill in the art would recognize that other food grade acidulants may also be used in the present invention. Alternatively, or in addition to, acidifying the quercetin-water mixture with food-grade acidulants, the mixture may also be acidified using fruit juices. Examples of fruit juices that may be used in accordance with the present invention include, but are not limited to, apple, grape, pear and citrus fruits in general.

The food grade acidulants may be added to the mixture prior to homogenization and/or after homogenization. The food grade acidulant is added in an amount to lower the pH to the desired pH level. For example, 0.1 to 0.2 wt %, based on total weight of the slurry.

In addition, a food grade preservative may be added to the mixture prior to homogenization and/or after homogenization. Suitable food grade preservatives include, but are not limited to, potassium sorbate, sodium benzoate, sodium hexametaphosphate, and EDTA. The preservative is added in an amount effective to preserve the slurry during storage. For example, 0.05 to 0.1 wt % based on total weight of the slurry.

Generally the acidulants, preservatives, and sequestrants are added to the water mixture prior to combining with the quercetin. This allows these ingredients to dissolve in the water before addition of the insoluble component.

Additional food-grade ingredients may also be used in accordance with the present invention. For example, colors, flavors, buffers, and stabilizers can be added to the slurry. One of ordinary skill in the art would appreciate that the list of food-grade ingredients set forth in the immediately preceding sentence is not all-inclusive and that other food-grade ingredients may also be used in the present invention.

Following formation of the quercetin-water mixture, the pH of the quercetin-water mixture may be adjusted with a food grade acidulant as discussed above. Typically, the pH is below about 6.5, such as about 2.5 to about 5.5, or about 2.5 to about 4.5. Generally the pH is about 3. Similarly, following formation of the quercetin slurry, the pH of the slurry may be adjusted with a food grade acidulant as discussed above. Typically, the pH is below about 6.5 about 2.5 to about 5.5, or about 2.5 to about 4.5, generally about 3.

A slurry is generally a thin mixture of a liquid, especially water, and finely divided insoluble substances. Viscosity of slurries can be evaluated with conventional equipment, i.e., rheometers or viscometers. The viscosity of the slurry is generally 5-100 cps as measured by a Brookfield viscometer.

In another aspect of the present invention, the slurry is added to beverages such as, but not limited to, ready-to-drink beverages, fruit juices, dairy beverages and carbonated soft drinks. This list is not all-inclusive and one of ordinary skill in the art would recognize that the slurry may be added to other beverages in accordance with the present invention.

In another aspect of the present invention, the slurry is added to foodstuff such as bars, chews, gels, gummies, strips, shots, and beverage concentrate.

Benefits of the present invention include providing a smooth product that remains stable for long periods of time. It is the intent to provide a product which maintains stability under normal distribution and storage conditions. As noted, homogenization is carried out under a total pressure of from about 3,000 to about 20,000 psi, generally with a total pressure of from about 5,000 to about 7,000 psi. The use of pressures lower than 3,000 psi may result in undesirable instability, whereas the use of pressures higher than 20,000 psi may be impractical. The use of a homogenization pressure of at least 3,000 psi ensures that the final composition possesses uniformity. The homogenization step may be carried out by any of the apparatus or methods conventional in the art. The number of passes may be one or two or more as is conventional in the art.

### Example 1

| | Amount |
|---|---|
| Ingredient | (% by wt.) |
| Beverage Water | 89-99 |
| Quercetin | 1-10 |
| Citric Acid | 0.1-0.4 |
| Potassium Sorbate | 0.03-0.12 |
| Total | 100.00 |

Add water to a tank and begin mixing. Add citric acid and mix until dissolved. Add potassium sorbate and mix until dissolved. Add quercetin (powder form) and mix for approximately 5 minutes. Homogenize at 6000 psi (first pass) and then again at 6000 psi (second pass). Mix slurry for 10 minutes. If necessary, adjust pH with additional citric acid to achieve a pH of 3.0.

### Example 2

| | Amount |
|---|---|
| Ingredient | (% by wt.) |
| Beverage Water | 94.7 |
| Quercetin | 5.0 |
| Citric Acid | 0.2 |
| Potassium Sorbate | 0.1 |
| Total | 100.00 |

Add water to a tank and begin mixing. Add citric acid and mix until dissolved. Add potassium sorbate and mix until dissolved. Add quercetin (powder form) and mix for approximately 5 minutes. Homogenize at 6000 psi (first pass) and then again at 6000 psi (second pass). Mix slurry for 10 minutes. If necessary, adjust pH with additional citric acid to achieve a pH of 3.0.

In addition to providing a stable slurry containing quercetin particles, the present process also provides a safer method of preparing microparticulated quercetin. Microparticularization of quercetin in the industry is generally performed by grinding or milling the particles into finer particles. However, this process is challenging because it is dusty (making it difficult to recover as much product), the particles are electrostatically charged, so they stick to the equipment and/or other processing tools, and they may be flammable.

As described, the present invention provides a method for preparing a quercetin slurry, with beneficial attributes and various applications in the food industry and other industries.

The invention may be embodied in other specific forms without departing from the spirit or essential characteristics thereof. The foregoing embodiments, therefore, are to be considered in all respects illustrative rather than limiting the invention described herein. Scope of the invention is thus indicated by the appended claims, rather than by the foregoing description, and all changes that come within the meaning and range of equivalency of the claims are intended to be embraced therein.

What is claimed is:

1. A method for preparing a quercetin slurry comprising the steps of:
a) combining water and quercetin;
b) mixing the water and quercetin to obtain a quercetin-water mixture; and
c) homogenizing the mixture to obtain a quercetin-water slurry, or mixing the mixture under high shear, ultrasonic processing, or both to obtain a quercetin-water slurry.

2. The method according to claim 1 comprising in step c) homogenizing the mixture to obtain a quercetin-water slurry.

3. The method of claim 1 further comprising in step a) combining at least one food-grade acidulant with the water and the quercetin.

4. The method of claim 3 wherein the at least one food-grade acidulant is selected from the group consisting of citric acid, ascorbic acid, phosphoric acid, lactic acid, malic acid, tartaric acid, and mixtures thereof.

5. The method of claim 1 further comprising in step a) combining at least one preservative with the water and the quercetin.

6. The method of claim 5 wherein the at least one preservative is selected from the group consisting of potassium sorbate, sodium benzoate, sodium hexametaphosphate, EDTA and mixtures thereof.

7. The method of claim 1 further comprising adjusting the pH of the quercetin-water mixture to below about 6.5.

8. The method of claim 7 wherein the pH is adjusted with a food grade acidulant.

9. The method of claim 1 wherein the viscosity of the slurry is 5-100 using a Brookfield viscometer.

10. The method of claim 1 further comprising in step a) adding at least one food-grade ingredient to the quercetin slurry, the at least one food-grade ingredient selected from the group consisting of stabilizers, colors, flavors and mixtures thereof.

11. The method of claim 1 wherein the quercetin comprises 2.5 to 10 wt % based on total weight of the slurry.

12. The method of claim 1 wherein the quercetin comprises about 2.5 to about 5 wt % based on total weight of the slurry.

13. The method of claim 1 wherein the average particle size of the quercetin is less than 20 microns.

14. The method of claim 1 wherein the average particle size of the quercetin is less than 3 micron.

15. A quercetin slurry prepared by
a) combining water and quercetin;
b) mixing the water and quercetin to obtain a quercetin-water mixture; and
c) homogenizing the mixture to obtain a quercetin-water slurry, or mixing the mixture under high shear, ultrasonic processing, or both to obtain a quercetin-water slurry.

16. A quercetin slurry comprising water and quercetin, wherein the average particle size of the quercetin is less than 20 microns.

17. The quercetin slurry of claim 16 further comprising a food grade acidulant.

18. The quercetin slurry of claim 17 wherein the at least one food-grade acidulant is selected from the group consisting of citric acid, ascorbic acid, phosphoric acid, lactic acid, malic acid, tartaric acid, and mixtures thereof.

19. The quercetin slurry according to claim 16 further comprising at least one preservative.

20. The quercetin slurry according to claim 19 wherein the preservative is selected from the group consisting of potassium sorbate, sodium benzoate, sodium hexametaphosphate, EDTA and mixtures thereof.

21. The quercetin slurry according to claim 16 wherein the pH of the quercetin-water mixture is below about 6.5.

22. The quercetin slurry according to claim 16 wherein the viscosity of the slurry is 5-100 using a Brookfield viscometer.

23. The quercetin slurry according to claim 16 further comprising at least one food-grade ingredient selected from the group consisting of stabilizers, colors, flavors and mixtures thereof.

24. The quercetin slurry according to claim 16 wherein the quercetin comprises 2.5 to 10 wt % based on total weight of the slurry.

25. The quercetin slurry according to claim 24 wherein the quercetin comprises about 2.5 to about 5 wt % based on total weight of the slurry.

26. The quercetin slurry according to claim 16 wherein the average particle size of the quercetin is less than 3 micron.
